(19) United States
(12) Patent Application Publication
(10) Pub. No.: US 2023/0002389 A1
(43) Pub. Date: Jan. 5, 2023

(54) ADENINE ANALOGS, PRODRUGS, DERIVATIVES, COMPOSITIONS AND USES THEREOF

(71) Applicant: Janak Khimchand Padia, Germantown, MD (US)

(72) Inventor: Janak Khimchand Padia, Germantown, MD (US)

(21) Appl. No.: 17/836,070

(22) Filed: Jun. 9, 2022

Related U.S. Application Data
(60) Provisional application No. 63/210,004, filed on Jun. 12, 2021.

(51) Int. Cl. C07D 473/34

(52) U.S. Cl. CPC C07D 473/34

(57) ABSTRACT

The present invention relates to adenine analogs, prodrugs thereof, derivatives thereof, compositions thereof, metabolites thereof, salts thereof, prodrugs thereof, and uses in pharmaceutical compositions containing these compounds, and methods of using these compounds for treating a wide variety of medical conditions, diseases or disorders thereof.

### FIELD OF THE INVENTION

The present invention relates to adenine analogs, prodrugs thereof, derivatives thereof, compositions thereof, metabolites thereof, salts thereof, prodrugs thereof, and uses in pharmaceutical compositions containing these compounds, and methods of using these compounds for treating a wide variety of medical conditions, diseases or disorders thereof.

### BACKGROUND OF THE INVENTION

Adenine is a nucleobase and id a purine derivative. Ribose derivative of adenine is called adenosine and it modulates many physiological processes. Cellular signaling by adenosine occurs through four known adenosine receptor (AR) subtypes (A1, A2A, A2B, and A3). ARs play an important role in physiology and pathophysiology and therefore represent attractive drug targets for a range of conditions. Cells of the immune system express these receptors and are responsive to the modulatory effects of adenosine in an inflammatory environment. Animal models of asthma, ischemia, arthritis, sepsis, inflammatory bowel disease, and wound healing have helped to elucidate the regulatory roles of the various adenosine receptors in dictating the development and progression of the disease. Modulation of adenosine receptors have many therapeutic effects in cardiovascular disease (for example: arrhythmia (Zablocki, et al., Current topics in medicinal chemistry, 4, 839-854, (2004); Fraser, et al., Journal of Pharmacology and Experimental Therapeutics, 305, 225-231, (2003); Bayes, et al., Methods and findings in experimental and clinical pharmacology, 25, 831-55, (2003))., ischaemia (Shneyvays, et al., Cell calcium, 36, 387-396, (2004); Tracey, et al., Cardiovascular research, 40, 138-145, (1998); Mozzicato, et al., The FASEB journal, 18, 406-408, (2004); Schindler, et al., British journal of pharmacology, 144, 642-650, (2005))., vasodilation (Schindler, et al., British journal of pharmacology, 144, 642-650, (2005); Varani, et al., Circulation, 102, 285-289, (2000)).), nervous system disorders (for example: dementia and anxiety disorders (Maemoto, et al., Journal of pharmacological sciences, 96, 42-52, (2004))., pain (Sawynok, European journal of pharmacology, 347, 1-11, (1998); Johansson, et al., Proceedings of the National Academy of Sciences, 98, 9407-9412, (2001); Wu, et al., Pain, 113, 395-404, (2005))., Parkinson's disease (Xu, et al., Pharmacology & therapeutics, 105, 267-310, (2005); Hauser, et al., Neurology, 61, 297-303, (2003); Chen, et al., Journal of Neuroscience, 19, 9192-9200, (1999))., Ischaemia and neuroprotection (Yu, et al., Nature medicine, 10, 1081, (2004); Olsson, et al., European Journal of Neuroscience, 20, 1197-1204, (2004); Turner, et al., Proceedings of the National Academy of Sciences, 100, 11718-11722, (2003))., Sleep (Porkka-Heiskanen, et al., Science, 276, 1265-1268, (1997); Stenberg, et al., Journal of sleep research, 12, 283-290, (2003); Basheer, et al., Progress in neurobiology, 73, 379-396, (2004))., renal system disorders (Pingle, et al., Journal of Biological Chemistry, 279, 43157-43167, (2004); Gottlieb, et al., Circulation, 105, 1348-1353, (2002); Lee, et al., American Journal of Physiology-Renal Physiology, 286, F298-F306, (2004))., pulmonary disorders (Sun, et al., The Journal of clinical investigation, 115, 35-43, (2005); Holgate, British journal of pharmacology, 145, 1009-1015, (2005); Fozard, et al., European journal of pharmacology, 438, 183-188, (2002))., inflammatory disorders (Sitkovsky, et al., Annu. Rev. Immunol., 22, 657-682, (2004); Ohta, et al., Nature, 414, 916, (2001))., endocrine disorders (Dong, et al., Diabetes, Obesity and Metabolism, 3, 360-366, (2001); Harada, et al., Journal of medicinal chemistry, 44, 170-179, (2001))., cancer (Antonioli, et al., Nature Reviews Cancer, 13, 842, (2013); Adamson, et al., Pharmacology, 15, 84-89, (1977); Stagg, et al., Oncogene, 29, 5346, (2010))., visual disorders (Okamura, et al., Bioorganic & medicinal chemistry letters, 14, 3775-3779, (2004); Avila, et al., Investigative ophthalmology & visual science, 43, 3021-3026, (2002))..

Adenosine itself has been modified to generate ligands for ARs, and extensive structure-activity relationship (SAR) studies have probed. Of the major modifications, analogs with the N6- or 2-position of the adenine moiety and in the 3′-, 4′- or 5′-position of the ribose moiety have produced valuable agonist and antagonists for ARs. The crystal structure of the seven-transmembrane protein rhodopsin, supported by mutagenesis studies, has provided a good understanding of ligand recognition and insights into conformational dynamics. With this information, rational drug design and molecular modeling using have been used for design and produced potent and selective ligands of ARs (Kim, et al., Journal of medicinal chemistry, 46, 4847-4859, (2003); Tchilibon, et al., Journal of medicinal chemistry, 48, 1745-1758, (2005); Moro, et al., Current drug discovery technologies, 2, 13-21, (2005); Moro, et al., Medicinal research reviews, 26, 131-159, (2006); Gao, et al., Journal of medicinal chemistry, 45, 4471-4484, (2002)). Adenine derivatives have also shown antagonist activities for 5HT_{2B}R and 5HT_{2C}R (Tosh, et al., Journal of Medicinal Chemistry, 59, 11006-11026, (2016)). Some of the key adenine derivatives are mentioned below:

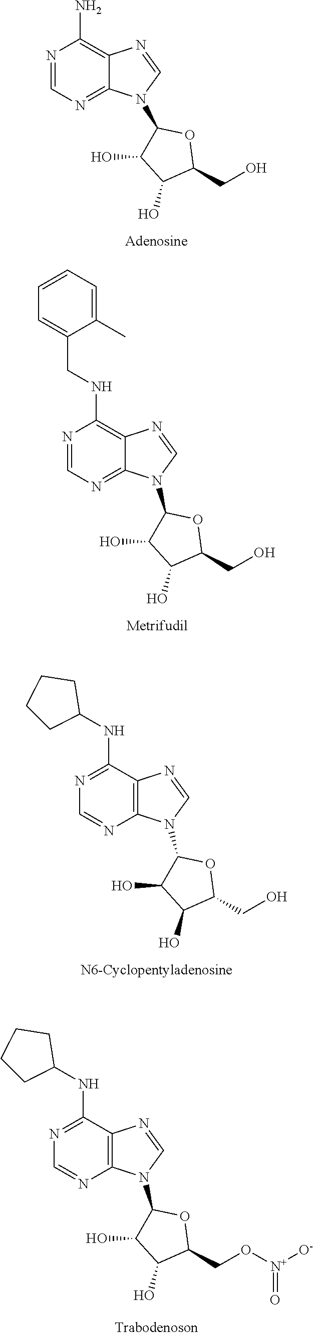 [SMILES: Nc1ncnc2c1ncn2[C@H]1O[C@@H](CO)[C@H](O)[C@@H]1O ; Cc1ccccc1CNc1ncnc2c1ncn2[C@H]1O[C@@H](CO)[C@H](O)[C@@H]1O ; OC[C@H]1O[C@@H](n2cnc3c(NC4CCCC4)ncnc32)[C@H](O)[C@@H]1O ; O=[N+]([O-])OC[C@@H]1O[C@H](n2cnc3c(NC4CCCC4)ncnc32)[C@@H](O)[C@H]1O]

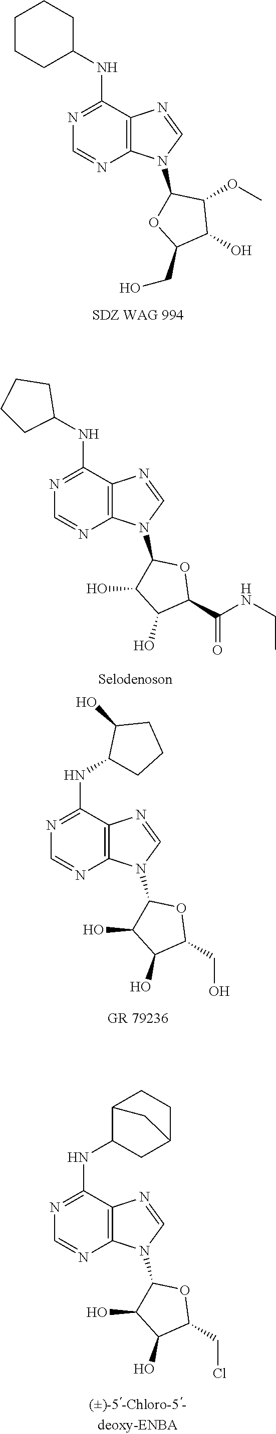 [SMILES: CO[C@@H]1[C@H](O)[C@@H](CO)O[C@H]1n1cnc2c(NC3CCCCC3)ncnc21 ; CCNC(=O)[C@@H]1O[C@H](n2cnc3c(NC4CCCC4)ncnc32)[C@@H](O)[C@H]1O ; OC[C@H]1O[C@@H](n2cnc3c(N[C@H]4CCC[C@@H]4O)ncnc32)[C@H](O)[C@@H]1O ; O[C@@H]1[C@H](O)[C@@H](CCl)O[C@H]1n1cnc2c(NC3CC4CCC3C4)ncnc21]

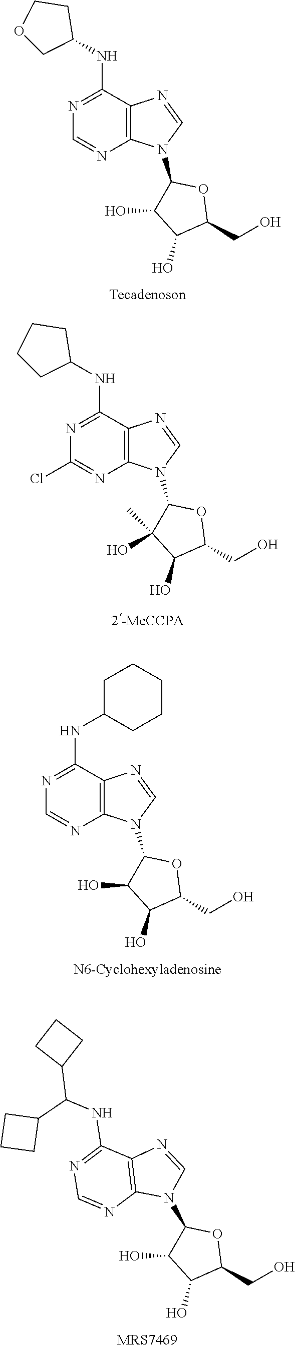 [SMILES: OC[C@@H]1O[C@H](n2cnc3c(N[C@H]4CCOC4)ncnc32)[C@@H](O)[C@H]1O ; C[C@@]1(O)[C@H](O)[C@@H](CO)O[C@H]1n1cnc2c(NC3CCCC3)nc(Cl)nc21 ; OC[C@H]1O[C@@H](n2cnc3c(NC4CCCCC4)ncnc32)[C@H](O)[C@@H]1O ; OC[C@@H]1O[C@H](n2cnc3c(NC(C4CCC4)C4CCC4)ncnc32)[C@@H](O)[C@H]1O]

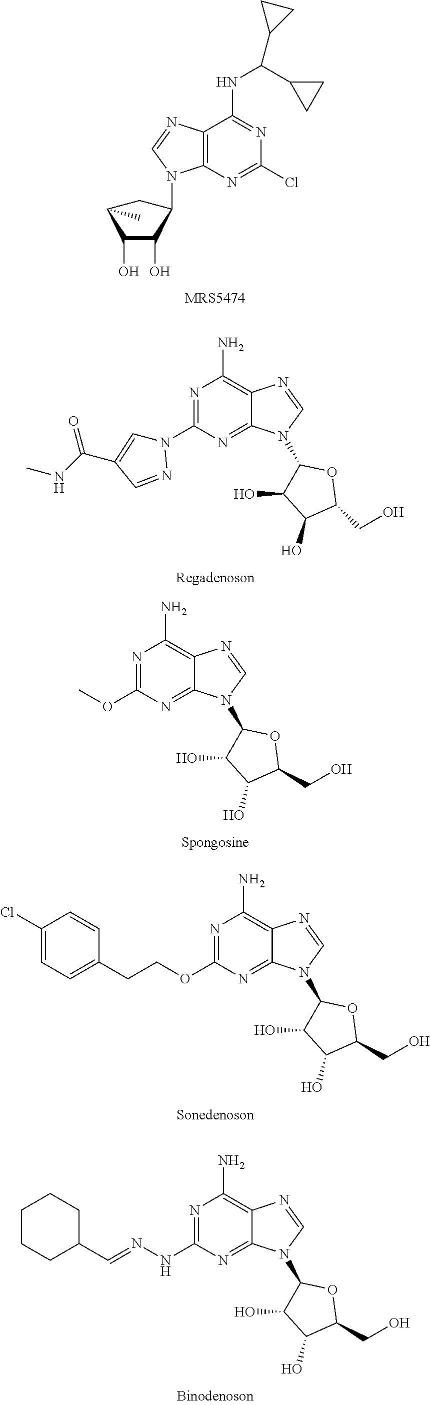

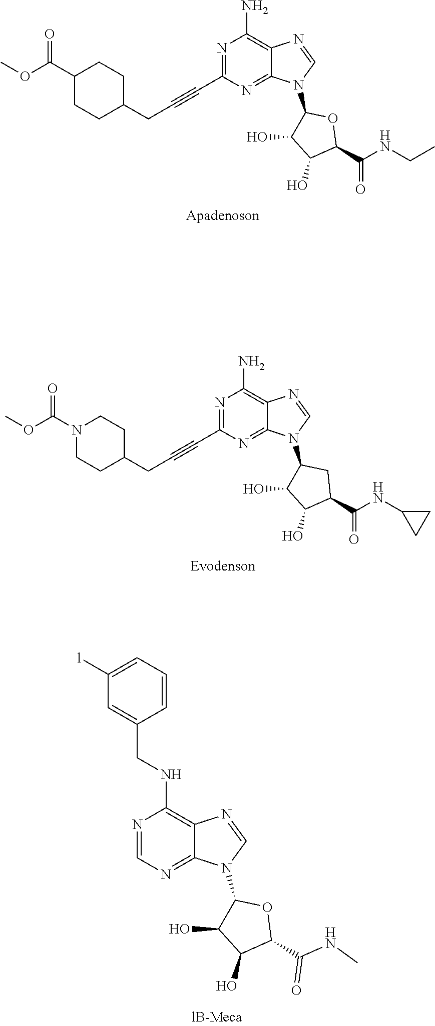

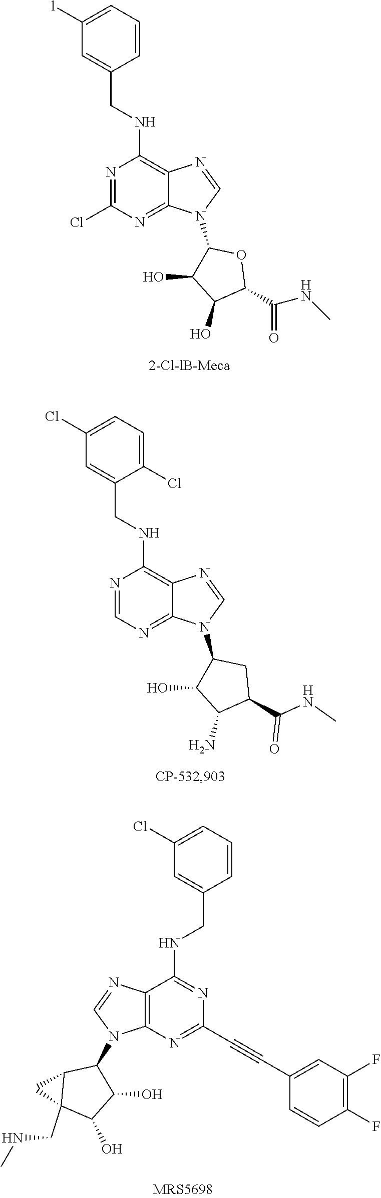

Adenine derivatives are reported as agonists, partial agonists, and antagonists for adenosine and other GPCRs. They have therapeutic benefits, including suppression of the immune response, glomerular filtration, seizures, stroke, depression, angina asthma, hypothermia pain, diabetes, obesity, cardiovascular, cancer, sickle cell disease, inflammation, liver diseases, erectile dysfunction, and dermatological conditions.

Several adenine derivatives have entered clinical trials; however, problems with their drug-like properties such as high lipophilicity, low water solubility, inadequate bioavailability, poor blood-brain barrier (BBB) penetration, and toxicity have limited their clinical development (Hess, et al., Journal of medicinal chemistry, 43, 4636-4646, (2000)). The compounds of this invention relate to designing and producing adenine derivatives with appropriate drug-like properties for therapeutic uses.

### SUMMARY OF THE INVENTION

The present invention relates to adenine analogs, salts thereof, prodrugs thereof, derivatives thereof, metabolites thereof, and uses in pharmaceutical compositions containing these compounds, and methods of using these compounds for treating a wide variety of medical conditions, diseases or disorders thereof.

In one aspect, a compound of Formula I or its pharmaceutically acceptable salt thereof or a prodrug thereof or metabolite thereof, or composition thereof

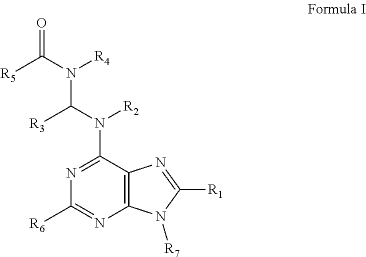

wherein

R₁ is selected from hydrogen, alkyl, alkenyl, alkynyl, aryl, heteroaryl wherein said alkyl, alkenyl, alkynyl, aryl, heteroaryl each are optionally substituted with one or more R₈;

R₂, R₅, and R₇ is selected from hydrogen, amino acid residue, alkyl, alkenyl, alkynyl, heteroalkyl, aryl, arylalkyl, heteroaryl, heteroarylalkyl, cycloalkyl, bicycloalkyl, and heterocycloalkyl wherein said amino acid, alkyl, alkenyl, alkynyl, heteroalkyl, aryl, arylalkyl, heteroaryl, heteroarylalkyl, cycloalkyl, bicycloalkyl, and heterocycloalkyl each are optionally substituted with one or more R₈;

R₃, and R₄ are independently from hydrogen and alkyl, wherein said alkyl is optionally substituted with one or more R₈;

R₆ is selected from hydrogen, halogen, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;

R₈ is independently selected for each occurrence from the group consisting of hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, amino, alkylamino, dialkylamino, diarylalkylamino, alkylthio, arylthio, heteroarylthio, oxo, oxa, —(CH₂)ₙOR₉, —C(O)—R₉, —C(O)OR₉, —C(O)NR₉R₁₀, —C(S)NR₉R₁₀, CO₂H, acyloxy, halo, —CN, —NO₂, —N₃, —SH, —OH, —CH(CF₃)NR₉R₁₀, —C(═N)NR₉, perhaloalkyl, perhaloalkoxy, perhaloacyl, guanidinyl, pyridinyl, thiophene, furanyl, indolyl, indazolyl, phosphonic acid, mono, or poly-phosphate derivative, phosphonate, phosphonic acid, phosphate, phosphoramide, sulfonate, sulfone, sulfate, sulphonamide, carbamate, urea, thiourea, thioamide, and thioalkyl; wherein R₉ and R₁₀ are independently selected from hydrogen, methyl, ethyl, and benzyl.

According to one embodiment, a pharmaceutical composition comprising a compound of Formula I or a pharmaceutically acceptable salt, ester, or prodrug or metabolite thereof in association with a pharmaceutically acceptable diluent or carrier to form a formulation system for delivering the compound.

According to one embodiment, a pharmaceutical composition comprising a compound of Formula I or a pharmaceutically acceptable salt, ester or prodrug or metabolite thereof in a combination of other pharmaceutically active agent(s).

According to one embodiment, a compound of Formula I or a pharmaceutically acceptable salt, ester, or prodrug or metabolite thereof for use in therapy.

According to one embodiment, the use of a compound of Formula I or a pharmaceutically acceptable salt, ester or prodrug or metabolite thereof in the preparation of a medicament for the treatment of cardiovascular disease, nervous system disorders, Parkinson's disease, neurodegenerative disorders, ischemia and neuroprotection, Sleep, renal system disorders, pulmonary disorders, inflammatory disorders, endocrine disorders, cancer, visual disorders.

According to one embodiment, a process of producing a compound of Formula I or its pharmaceutically acceptable salt or prodrug or metabolite.

According to another aspect, a method for the treatment or prevention of a disease or condition modulated by GPCR(s) is provided and includes the step of administering a compound as provided herein. Any of the methods or uses provided herein may include administering to a subject a therapeutically effective amount of a compound as provided herein, including salt or polymorph thereof, or a pharmaceutical composition that includes such compounds.

The invention relates to formulation systems loaded with compounds of this invention mention above which said systems have improved biopharmaceutical properties for solubility, drug concentration in the target tissue(s), in vivo efficacy and safety, improved quality (fineness and homogeneity of the particles, drug inclusion) and improved physical stability of the particulate formulation (no aggregation or gel formation). The compound of this invention can be appropriately formulated for desired delivery systems such as oral drug delivery (immediate release, delayed-release, prolonged-release, modified release), parenteral drug delivery, ophthalmic drug delivery, nasal drug delivery, rectal drug delivery, colon-specific drug delivery, topical drug delivery, and CNS or brain drug delivery by powder injection; or by buccal, sublingual, or intranasal absorption. Pharmaceutical compositions may be formulated in unit dose form or multiple or subunit doses.

The manner in which the compounds or their pharmaceutical composition set forth herein may be administered can vary. According to one embodiment, the compounds can be administered orally. Preferred pharmaceutical compositions may be formulated for oral administration in the form of tablets, capsules, caplets, syrups, solutions, and suspensions. Such oral formulations can be provided in modified release dosage forms such as time-release tablets and capsule formulations, including enteric tablets and enteric capsules. Pharmaceutical compositions can also be administered via injection, namely, intravenously, intramuscularly, subcutaneously, intraperitoneally, intra-arterial, intrathecally, and intracerebroventricularly.

Suitable carriers for injection are well known to those of skill in the art and include 5% dextrose solutions, saline, and phosphate-buffered saline.

According to another aspect, a formulation of the compound of the present invention can be prepared by entrapping the compound in liposomes, or albumin. The formulation of the compound of the present invention can be prepared by using nanoparticles, nanocapsules or nanospheres using appropriate excipient(s) such as cyclodextrins, mannitol, sodium dodecyl sulfate, albumin, polysorbate 80, trehalose, sucrose, lactose, tromethamine, sodium chloride, gelatin, amino acids.

In another embodiment of the invention, stabilizing excipients for preparing formulations of a compound of Formula I are selected from hydrophobicity inducing agents. These agents may be represented by magnesium Stearate, Stearic acid, glyceryl Stearate, glyceryl palmitostearate, Stearoyl macrogolglycerides, lauroyl macrogolglycerides, waxes, and hydrogenated vegetable oils, among others.

The stabilizers may be included into the formulations for a compound of Formula I for the of the current invention in the amount Such that, for an individual stabilizer, the ratio of the parts by weight of stabilizer to parts by weight of the drug substance is from 0.1:1 to 50:1, preferably from 0.25:1 to 40:1; most preferably from 0.4:1 to 25:1. Combinations of stabilizing excipients may be used in all embodiments of the instant invention and may provide synergistic stabilizing action.

Stabilizers may be incorporated into formulations of a compound of Formula I in a variety of ways. They may be intermixed with the drug Substance and/or other excipients or may be provided in the form of a coating on the compound of Formula I-containing substrate. Water-based acidifiers may be used in the preparation of the formulations of the current invention as long as care is taken to eliminate or reduce water during the processing. Alternatively, excipients, such as bulking agents, maybe pre-treated by the stabilizers prior to their incorporation into the formulation. Stabilization of compound of Formula I may also be achieved by coating drug layered Substrates with coating polymers dissolved or dispersed in acidic Solution. These and further ways of using stabilizers are disclosed in more detail in the examples below. Additional excipients that can be used alone or in combination to formulate a stable compound of Formula I drug products in accordance with the current invention include bulking agents. Such as lactose anhydrous or lactose monohydrate, (i.e., Supertab 21AN, Ludipress, Ludipress LCE, Fast Flo Lactose, Supertose, Pharmatose, Respitose), glyceryl behenate, hypromellose, ascorbic acid, benzoic acid, carbomer, low moisture microcrystalline cellulose (Avicel® grades PH-103, PH-112, PH-113, PH-200), colloidal silicon dioxide, dextrose (anhydrous), dextrose (anhydrous), maltol, fructose, glyceryl palmitostearate, glyceryl monostearate, guar gum, lactitol (anhydrous), magnesium carbonate, maltitol, maltose, mannitol, polyethylene oxide, Sorbitol. Sucrose, compressible Sugar, confectioner's Sugar, Xylitol; glidants such as talc, starch, and colloidal silicon dioxide and the metallic Stearates; lubricants selected from talc, sodium Stearyl fumarate, hydrogenated vegetable oils, glyceryl palmitostearate, glyceryl behenate, poloxamer, Stearic acid, Stearyl alcohol, cetyl alcohol, waxes, and the metallic Stearates; wetting and solubility enhancing agents, such as sodium lauryl sulfate, polyethylene glycol, PEG glyceryl esters, lecithin, poloxamer, the polysorbates, the polyoxyethylene alkyl ethers, polyethylene castor oil derivatives, polyethylene Stearate, and the Sorbitan esters. Through the use of stabilizers and low levels of moisture as described above, the inventors were able to realize one goal of the current invention: to provide stable IR formulations of a compound of Formula I that comprise not more than 5% of water. In yet further embodiment, the invention discloses stable IR formulations of a compound of Formula I comprising stabilizing excipients. A further goal of the current invention is to utilize stabilization techniques described herein to provide stable MR formulations of a compound of Formula I comprising an active compound, at least one release controlling polymer that may be a non-pH-dependent polymer or a pH-dependent, enteric polymer, and at least one pharmaceutically acceptable excipient.

Further, the invention provides MR formulations of a compound of Formula I comprising a compound of Formula I, at least one release controlling polymer and at least one pharmaceutically acceptable excipient, wherein the total amount of residual water in the formulation is not more than 5% by weight of the formulation. The MR formulations of a compound of Formula I exhibiting XR profile, or combination of XR and DR profile, or any combination of those with IR profile are disclosed herein. These specific release profiles are achieved by formulating compound of Formula I, at least one release controlling polymer and one or more excipient in a variety of inventive formulations. The release controlling polymers of the current invention may be selected from non-pH-dependent polymers such as hydrophilic rate controlling compound that can be used to formulate MR multi-particulates or matrix tablets drug products, and hydrophobic rate-controlling compounds that exhibit limited or no water solubility; or enteric polymers that exhibit pH-dependent solubility.

Osmotic tablets can be formulated as a single or as a multiple layer core. In one embodiment, the osmotic tablet comprises a bilayer core, wherein one layer comprises agents to modulate drug release, such as a solubilizer, that are released in a Sustained manner, and the second layer comprises the drug and potentially other agents to modulate drug release. Stabilizers listed above may be contained in at least one layer of the osmotic formulation. An overcoat of a drug can be applied to the osmotic tablet following a functional coating to provide an immediate release component to the dosage form. Alternatively, the osmotic tablet may be coated with an enteric polymer on top of the semipermeable rate-controlling membrane providing a DR/XR profile.

Pharmaceutical compositions may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation); by powder injection; or by buccal, sublingual, or intranasal absorption. Pharmaceutical compositions may be formulated in unit dose form, or multiple or subunit doses.

The administration of the pharmaceutical compositions described herein can be intermittent or at a gradual, continuous, constant, or controlled rate. The pharmaceutical compositions may be administered to a warm-blooded animal, for example, a mammal such as a human being. In addition, the time of day and the number of times per day that the pharmaceutical composition is administered can vary.

The compounds, as provided herein, may also be used for the preparation of a medicament for the treatment or prevention of a disease or condition modulated by GPCR(s). Methods for treating, preventing, delaying the onset of, or slowing the progression of disorders mediated by GPCR(s) involved in the regulation or dysregulation of gene expression in mammals in need of such treatment are also provided. The methods involve administering to a subject a therapeutically effective amount of a compound as provided herein, including a salt thereof, or a pharmaceutical composition that includes such compounds.

According to one embodiment, the methods for treating, preventing, delaying the onset of, or slowing the progression of disorders mediated by acetylated proteins involved in the regulation or dysregulation of gene expression, in mammals in need of such treatment include the administration of at least one compound as provided herein including, but not limited to, the compounds provided according to Formula I.

The compounds alone or in a pharmaceutical composition as provided herein may be used in the treatment of a variety of disorders and conditions and, as such, may be used in combination with a variety of other suitable therapeutic agents useful in the treatment or prophylaxis of those disorders or conditions. Thus, one embodiment of the present disclosure includes the administration of the compound of the present disclosure in combination with other therapeutic compounds. Such a combination of pharmaceutically active agents may be administered together or separately and, when administered separately, the administration may occur simultaneously or sequentially, in any order. The amounts of the compounds or agents and the relative timings of administration will be selected in order to achieve the desired therapeutic effect. The administration in a combination of a compound of the present disclosure with other treatment agents may be in combination by administration concomitantly in (1) a unitary pharmaceutical composition including two or more compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present disclosure includes combination therapy comprising administering to the subject a therapeutically or prophylactically effective amount of the compound of the present disclosure and one or more other therapy including chemotherapy, radiation therapy, gene therapy, or immunotherapy.

### DETAILED DESCRIPTION OF THE INVENTION

The present invention relates to adenine analogs, salts thereof, prodrugs thereof, derivatives thereof, metabolites thereof, and uses in pharmaceutical compositions containing these compounds, and methods of using these compounds for treating a wide variety of medical conditions, diseases or disorders thereof.

In one aspect, a compound of Formula I or its pharmaceutically acceptable salt thereof or a prodrug thereof or metabolite thereof, or composition thereof

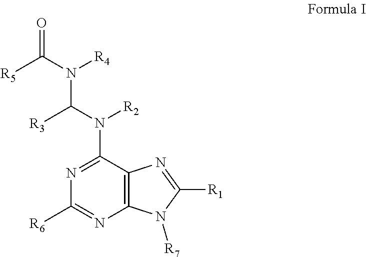

wherein

R₁ is selected from hydrogen, alkyl, alkenyl, alkynyl, aryl, heteroaryl wherein said alkyl, alkenyl, alkynyl, aryl, heteroaryl each are optionally substituted with one or more R₈;

R₂, R₅, and R₇ is selected from hydrogen, amino acid residue, alkyl, alkenyl, alkynyl, heteroalkyl, aryl, arylalkyl, heteroaryl, heteroarylalkyl, cycloalkyl, bicycloalkyl, and heterocycloalkyl wherein said amino acid, alkyl, alkenyl, alkynyl, heteroalkyl, aryl, arylalkyl, heteroaryl, heteroarylalkyl, cycloalkyl, bicycloalkyl, and heterocycloalkyl each is optionally substituted with one or more R₈;

R₃ and R₄ are independently from hydrogen and alkyl, wherein said alkyl is optionally substituted with one or more R₈;

R₆ is selected from hydrogen, halogen, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;

R₈ is independently selected for each occurrence from the group consisting of hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, amino, alkylamino, dialkylamino, diarylalkylamino, alkylthio, arylthio, heteroarylthio, oxo, oxa, —(CH₂)ₙOR₉, —C(O)—R₉, —C(O)OR₉, —C(O)NR₉R₁₀, —C(S)NR₉R₁₀, CO₂H, acyloxy, halo, —CN, —NO₂, —N₃, —SH, —OH, —CH(CF₃)NR₉R₁₀, —C(═N)NR₉, perhaloalkyl, perhaloalkoxy, perhaloacyl, guanidinyl, pyridinyl, thiophene, furanyl, indolyl, indazolyl, phosphonate, phosphonic acid, phosphate, phosphoramide, sulfonate, sulfone, sulfate, sulphonamide, carbamate, urea, thiourea, thioamide, and thioalkyl; wherein R₉ and R₁₀ are independently selected from hydrogen, methyl, ethyl, and benzyl.

According to one embodiment, the invention also provides a compound of Formula I wherein R₁ is hydrogen.

According to one embodiment, the invention also provides a compound of Formula I wherein R₂ is selected from hydrogen, alkyl, heteroalkyl, arylalkyl, heteroaryl, heteroarylalkyl, cycloalkyl, bicycloalkyl, and heterocycloalkyl wherein said alkyl, heteroalkyl, arylalkyl, heteroaryl, heteroarylalkyl, cycloalkyl, bicycloalkyl, and heterocycloalkyl each are optionally substituted with one or more R₈.

According to one embodiment, the invention also provides a compound of Formula I wherein R₃ is independently selected from hydrogen, methyl, ethyl, isopropyl, and phenyl.

According to one embodiment, the invention also provides a compound of Formula I wherein R₄ is selected from hydrogen and alkyl, wherein said alkyl is optionally substituted with R₈.

According to one embodiment, the invention also provides a compound of Formula I wherein R₅ is selected amino acid residue, alkyl, heteroalkyl, aryl, arylalkyl, heteroaryl, heteroarylalkyl, wherein said amino acid residue, alkyl, heteroalkyl, aryl, arylalkyl, heteroaryl, heteroarylalkyl, each is optionally substituted with one or more R₈.

According to one embodiment, the invention also provides a compound of Formula I wherein R₆ is selected from hydrogen, halogen, alkyl, aryl, heteroaryl, and alkynyl-aryl wherein said alkyl, aryl, heteroaryl, alkynyl-aryl each is optionally substituted with one or more R₈.

According to one embodiment, the invention also provides a compound of Formula 1 or wherein R₆ is selected from Formula IIA and Formula IB

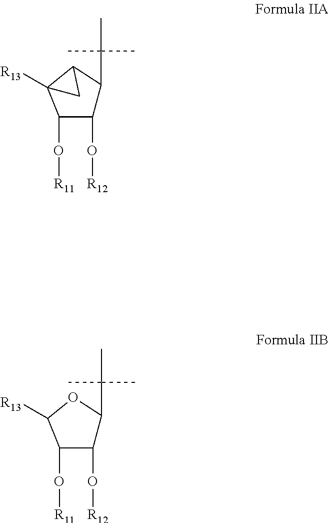

wherein

R₁₁ and R₁₂ are independently selected from hydrogen, alkyl, amino acid residue, —C(O)R₁₄, —C(O)NR₁₄R₁₅ wherein R₁₄, and R₁₅ are independently selected from hydrogen, alkyl wherein said alkyl and aryl each are optionally substituted with one or more R₈; and R₁₃ is selected from hydrogen, —CH₂—OH, —CH₂—Cl, —COOR₁₆, CONR₁₆R₁₇ wherein R₁₆ and R₁₇ are independently selected from hydrogen, alkyl wherein said alkyl and aryl each are optionally substituted with one or more R₈.

According to one embodiment, the invention also provides a compound of Formula I wherein R₆ is selected from Formula IIIA and Formula IIIB.

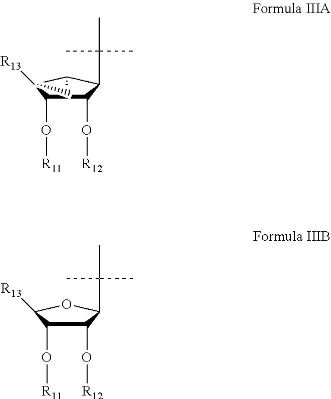

According to one embodiment, the invention also provides a compound of Formula I wherein R₆ is Formula IIIA.

According to one embodiment, the invention also provides a compound of Formula I wherein R₆ is Formula IIIB.

According to one embodiment, the invention also provides a compound of Formula I having the structure of Formula IV:

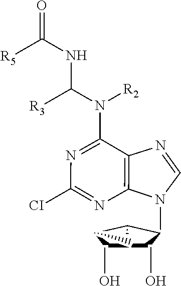

According to one embodiment, the invention also provides a compound of Formula I having the structure of Formula V:

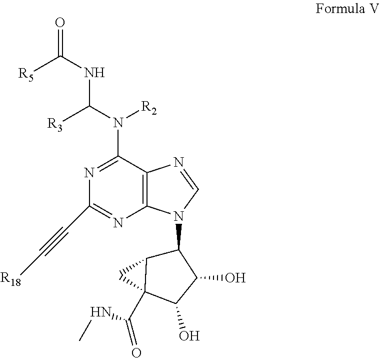

According to one embodiment, the invention also provides a compound of Formula I having the structure of Formula VIA:

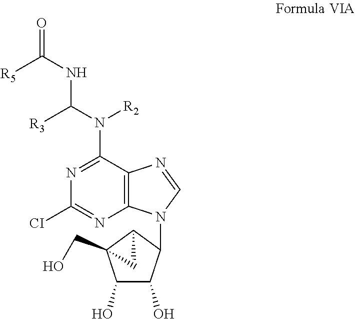

According to one embodiment, the invention also provides a compound of Formula I having the structure of Formula VIB:

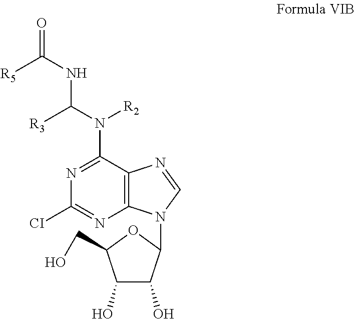

According to one embodiment, the invention also provides a compound of Formula I having the structure of Formula VII:

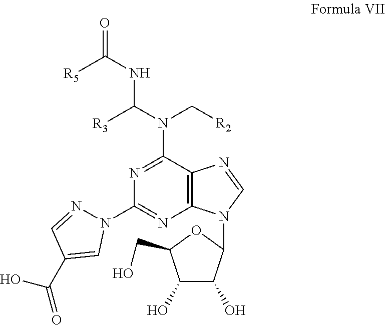

According to one embodiment, the invention also provides a compound of Tables 1-4 or its pharmaceutically acceptable salt thereof or a prodrug thereof or metabolite thereof, or composition thereof.

According to one embodiment, the invention also provides a pharmaceutical composition comprising a compound of Formula I as defined in claim 1 or a pharmaceutically acceptable salt, ester, or prodrug or metabolite thereof in association with a pharmaceutically acceptable diluent or carrier.

According to one embodiment, the invention also provides a pharmaceutical composition comprising a compound of Formula I as defined in claim 1 or a pharmaceutically acceptable salt, ester or prodrug, or metabolite thereof in association with a pharmaceutically acceptable diluent or carrier to form a formulation system for delivering the compound.

According to one embodiment, the invention also provides a pharmaceutical composition comprising a compound of Formula I as defined in claim 1 or a pharmaceutically acceptable salt, ester, or prodrug or metabolite thereof in a combination of other pharmaceutically active agent(s).

According to one embodiment, the invention also provides a compound of Formula I as defined in claim 1 or a pharmaceutically acceptable salt or a prodrug or metabolite thereof for use in therapy.

According to one embodiment, the invention also provides use of a compound of Formula I as defined in claim 1 or a pharmaceutically acceptable salt, ester or prodrug or metabolite thereof in the preparation of a medicament for the treatment of cardiovascular disease, nervous system disorders, Parkinson's disease, pain, neurodegenerative disorders, ischemia and neuroprotection, sleep, renal system disorders, pulmonary disorders, inflammatory disorders, endocrine disorders, cancer, and visual disorders.

According to one embodiment, the invention also provides a process of producing a compound of Formula I as defined in claim 1 or its pharmaceutically acceptable salt or prodrug or metabolite.

According to one embodiment, the invention also provides a modified-release formulation comprising a compound of Formula I a single active pharmaceutical ingredient, (a) at least one release controlling polymer selected from the group consisting of pH-dependent polymers and non-pH-dependent polymers, and (b) at least one stabilizer selected from the group consisting of acidifying agents and hydrophobizing agents, wherein the total amount of water content in the formulation is not more than 5% by weight of the formulation.

According to one embodiment, a pharmaceutical composition comprising a compound of Formula I or a pharmaceutically acceptable salt or a prodrug or metabolite thereof in association with a pharmaceutically acceptable diluent or carrier.

According to one embodiment, a pharmaceutical composition comprising a compound of Formula I or a pharmaceutically acceptable salt or a prodrug or metabolite thereof in association with a pharmaceutically acceptable diluent or carrier to form a formulation system for delivering the compound.

According to one embodiment, a pharmaceutical composition comprising a compound of Formula I as defined in claim 1 or a pharmaceutically acceptable salt or a prodrug or metabolite thereof in a combination of other pharmaceutically active agent(s).

According to one embodiment, a compound of Formula I or a pharmaceutically acceptable salt or a prodrug or metabolite thereof for use in therapy.

According to one embodiment, the use of a compound of Formula I or a pharmaceutically acceptable salt, ester or prodrug or metabolite thereof in the preparation of a medicament for the treatment of cardiovascular disease, nervous system disorders, Parkinson's disease, neurodegenerative disorders, ischemia and neuroprotection, Sleep, renal system disorders, pulmonary disorders, inflammatory disorders, endocrine disorders, cancer, visual disorders.

According to one embodiment, a process of producing a compound of Formula I or its pharmaceutically acceptable salt or prodrug or metabolite.

According to another aspect, a method for the treatment or prevention of a disease or condition by administrating a compound of Formula I. Any of the methods or uses provided herein may include administering to a subject a therapeutically effective amount of a compound as provided herein, including salt or polymorph, or prodrug thereof, or a pharmaceutical composition that includes such compounds.

The invention relates to formulation systems loaded with compounds of this invention mention above which said systems have improved biopharmaceutical properties for solubility, drug concentration in the target tissue(s), in vivo efficacy and safety, improved quality (fineness and homogeneity of the particles, drug inclusion) and improved physical stability of the particulate formulation (no aggregation or gel formation). The compound of this invention can be appropriately formulated for desired delivery systems such as oral drug delivery (immediate release, delayed-release, prolonged-release, modified release), parenteral drug delivery, ophthalmic drug delivery, nasal drug delivery, rectal drug delivery, colon-specific drug delivery, topical drug delivery, and CNS or brain drug delivery by powder injection; or by buccal, sublingual, or intranasal absorption. Pharmaceutical compositions may be formulated in unit dose form or multiple or subunit doses.

The manner in which the compounds or their pharmaceutical composition set forth herein may be administered can vary. According to one embodiment, the compounds can be administered orally. Preferred pharmaceutical compositions may be formulated for oral administration in the form of tablets, capsules, caplets, syrups, solutions, and suspensions. Such oral formulations can be provided in modified release dosage forms such as time-release tablet and capsule formulations, including enteric tablets and enteric capsules. Pharmaceutical compositions can also be administered via injection, namely, intravenously, intramuscularly, subcutaneously, intraperitoneally, intra-arterial, intrathecally, and intracerebroventricularly.

Suitable carriers for injection are well known to those of skill in the art and include 5% dextrose solutions, saline, and phosphate-buffered saline.

According to another aspect, a formulation of the compound of the present invention can be prepared by entrapping the compound in liposomes, or albumin. The formulation of the compound of the present invention can be prepared by using nanoparticles, nanocapsules or nanospheres using appropriate excipient(s) such as cyclodextrins, mannitol, sodium dodecyl sulfate, albumin, polysorbate 80, trehalose, sucrose, lactose, tromethamine, sodium chloride, gelatin, amino acids.

Pharmaceutical compositions may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation); by powder injection; or by buccal, sublingual, or intranasal absorption. Pharmaceutical compositions may be formulated in unit dose form or multiple or subunit doses.

The administration of the pharmaceutical compositions described herein can be intermittent, or at a gradual, continuous, constant, or controlled rate. The pharmaceutical compositions may be administered to a warm-blooded animal, for example, a mammal such as a human being. In addition, the time of day and the number of times per day that the pharmaceutical composition is administered can vary.

The compounds, as provided herein, may also be used for the preparation of a medicament for the treatment or prevention of a disease or condition modulated by GPCR(s). Methods for treating, preventing, delaying the onset of, or slowing the progression of disorders mediated by GPCR(s) involved in the regulation or dysregulation of gene expression in mammals in need of such treatment are also provided. The methods involve administering to a subject a therapeutically effective amount of a compound as provided herein, including a salt thereof, or a pharmaceutical composition that includes such compounds.

According to one embodiment, the methods for treating, preventing, delaying the onset of, or slowing the progression of disorders mediated by acetylated proteins involved in the regulation or dysregulation of gene expression, in mammals in need of such treatment include the administration of at least one compound as provided herein including, but not limited to, the compounds provided according to Formula I.

The compounds alone or in a pharmaceutical composition as provided herein may be used in the treatment of a variety of disorders and conditions and, as such, may be used in combination with a variety of other suitable therapeutic agents useful in the treatment or prophylaxis of those disorders or conditions. Thus, one embodiment of the present disclosure includes the administration of the compound of the present disclosure in combination with other therapeutic compounds. Such a combination of pharmaceutically active agents may be administered together or separately and, when administered separately, the administration may occur simultaneously or sequentially, in any order. The amounts of the compounds or agents and the relative timings of administration will be selected in order to achieve the desired therapeutic effect. The administration in a combination of a compound of the present disclosure with other treatment agents may be in combination by administration concomitantly in (1) a unitary pharmaceutical composition including two or more compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present disclosure includes combination therapy comprising administering to the subject a therapeutically or prophylactically effective amount of the compound of the present disclosure and one or more other therapy including chemotherapy, radiation therapy, gene therapy, or immunotherapy.

### Definitions

The following definitions are meant to clarify, but not limit, the terms defined. If a particular term used herein is not specifically defined, such a term should not be considered indefinite. Rather, terms are used within their accepted meanings.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein, the term “alkyl” refers to a straight or branched chain hydrocarbon, which may be optionally substituted, with multiple degrees of substitution being allowed. The term “lower alkyl” refers to an alkyl that includes from one to six carbon atoms, Examples of “lower alkyl” as used herein include, but are not limited to, methyl, ethyl, propyl, isopropyl, isobutyl, n-butyl, tert-butyl, isopentyl, and n-pentyl.

As used herein, the term “alkene” or “alkenyl” group refers to an unsaturated hydrocarbon that includes one or more carbon-carbon double bonds. The term “lower alkene” refers to an alkene that includes from two to twenty carbon atoms, such as from two to ten carbon atoms. The term “substituted alkene” refers to an alkene that has one or more of its hydrogen atoms replaced by one or more substituent groups, such as halogen.

As used herein, the term “alkyne” or “alkynyl” group refers to an unsaturated hydrocarbon that includes one or more carbon-carbon triple bonds. The term “lower alkyne” refers to an alkyne that includes from two to twenty carbon atoms, such as from two to ten carbon atoms. The term “substituted alkyne” refers to an alkyne that has one or more of its hydrogen atoms replaced by one or more substituent groups, such as halogen.

As used herein, the term “cycloalkyl” refers to a fully saturated optionally substituted monocyclic, bicyclic, or bridged hydrocarbon ring, with multiple degrees of substitution being allowed. Preferably, the ring is three to twelve-membered, more preferably, from five- to six-membered. Exemplary “cycloalkyl” groups as used herein include, but are not limited to, cyclopropyl, cyclobutyl, cyclopentyl, cyclohexyl, and cycloheptyl.

As used herein, the term “alkoxy” refers to a group —OR^{a}, where R^{a} is “alkyl” as defined herein.

As used herein, the term “heterocycloalkyl” or “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and carbon oxides. Preferably, the ring is three to twelve-membered, more preferably four, five or six-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, tetrahydrothiophene, pyrrolidinone, dihydrofuranone, thiazolidinone, azetidinone, cyclopentanone, piperidinone, thiomorpholinone, 2H-1,4-thiazin-3(4H)-one, dihydropyrimidine-2,4(1H,3H)-dione 1,4-dihydropyridine.

As used herein, the term “aryl” refers to a single benzene ring or fused benzene ring system, which may be optionally substituted, with multiple degrees of substitution being allowed. Examples of “aryl” groups as used include, but are not limited to, phenyl, benzyl, 2-naphthyl, 1-naphthyl, anthracene, and phenanthrene. Preferable aryl rings have five- to ten-members. The term “aryl” also includes a fused benzene ring system, namely where a cyclic hydrocarbon or heterocycle (e.g., a cyclohexane or dioxane ring) or heteroaryl (e.g., pyridine) is fused with an aromatic ring (aryl, such as a benzene ring).

As used herein, the term “heteroaryl” refers to a monocyclic five to seven-membered aromatic ring, a fused bicyclic aromatic ring system comprising two of such aromatic rings, which may be optionally substituted, with multiple degrees of substitution being allowed, or to a fused bicyclic ring system namely where a cycloalkyl or heterocycle (e.g., a cyclohexane or dioxane ring) is fused with a heteroaryl ring. Preferably, heteroaryl rings contain five- to ten-members. These heteroaryl rings contain one or more nitrogen, sulfur, and/or oxygen atoms. In certain embodiments, the heteroaryl rings contain one to three nitrogen, one to three oxygen, or one or two sulfur atoms. N-oxides, sulfur oxides and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, triazole, pyridine, pyridazine, pyrazine, pyrimidine, quinoline, isoquinoline, quinoxaline, benzofuran, benzoxazole, benzothiophene, indole, indazole, benzimidazole, imidazopyridine, pyrazolopyridine, and pyrazolopyrimidine.

As used herein, the term “halogen” refers to fluorine, chlorine, bromine, or iodine.

As used herein, the term “haloalkyl” refers to a substituted or unsubstituted alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein, the term “sulfhydryl” refers to refers to a —SH group.

As used herein, the term “alkylthio” and thioalkyl refers to a group —SR^{a}, where R^{a} is “alkyl” as defined herein.

As used herein, the term “arylthio” refers to a group —SR^{a}, where R^{a} is “aryl” as defined herein.

As used herein, the terms “carboxyamido” and “amido” refers to —NR^{a}—C(O)—W or —C(O)—NR^{a}R^{b} wherein W is hydrogen or an unsubstituted or substituted alkyl, alkene, alkyne, cycloalkyl, aryl, or heterocycle group, or R^{a} and R^{b} can form cycloalkyl or heterocycle.

As used herein, the term “amine” is given its ordinary meaning and includes primary, secondary, and tertiary amines.

As used herein, the term “amido” refers to a group of the formula —C(O)NR^{a}R^{b}, wherein R^{a} and R^{a} are substituted, or unsubstituted alkyl, cycloalkyl or heterocycle, or R^{a} and R^{b} can form cycloalkyl or heterocycle.

As used herein, the term “sulfamido” refers to the group —SO₂—NR^{a}R^{b} wherein R^{a} and R^{a} are substituted, or unsubstituted alkyl, cycloalkyl or heterocycle, or R^{a} and R^{b} can form cycloalkyl or heterocycle.

As used herein, “optionally substituted”, groups may be substituted or unsubstituted. The substituent (or substitution) group may include, without limitation, one or more substituents independently selected from the following groups or designated subsets thereof: lower (C₁-C₆) alkyl, lower alkenyl, lower alkynyl, lower aryl, heteroaryl, alicyclic, heterocyclic, arylalkyl, heteroarylalkyl, lower alkoxy, lower aryloxy, amino, alkylamino, dialkylamino, diarylalkylamino, alkylthio, arylthio, heteroarylthio, oxo, oxa, carbonyl (—C(O)), carboxy esters (—C(O)OR). carboxamido (—C(O)NH₂), carboxy, acyloxy, —H, halo, —CN, —NO₂, —N₃, —SH, —OH. —C(O)CH₃, perhaloalkyl, perhaloalkoxy, perhaloacyl, guanidine, pyridinyl, thiophene, furanyl, indole, indazole, esters, amides, mono or poly-phosphonate derivative, phosphonic acid, phosphates, phosphoramides, sulfonates, sulfones, sulfates, sulphonamides, carbamates, ureas, thioureas and thioamides, thioalkyls. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

As used herein, the term “pharmaceutically acceptable” refers to the carrier(s), diluent(s), excipient(s) or salt forms of the compounds of the present disclosure that are compatible with the other ingredients of the formulation of the pharmaceutical composition.

As used herein, the term “pharmaceutical composition” refers to a compound of the present disclosure optionally admixed with one or more pharmaceutically acceptable carriers, diluents, or excipients. Pharmaceutical compositions preferably exhibit a degree of stability to environmental conditions to make them suitable for manufacturing and commercialization purposes.

As used herein, the terms “effective amount”, “therapeutic amount”, and “effective dose” refer to an amount of the compound of the present disclosure sufficient to elicit the desired pharmacological or therapeutic effects, thus resulting in effective prevention or treatment of a disorder. Treatment of a disorder may be manifested by delaying or preventing the onset or progression of the disorder, as well as delaying or preventing the onset or progression of symptoms associated with the disorder. Treatment of a disorder may also be manifested by a decrease or elimination of symptoms, reversal of the progression of the disorder, as well as any other contribution to the well-being of the patient. The effective dose can vary, depending upon factors such as the condition of the patient, the severity of the symptoms of the disorder, and the manner in which the pharmaceutical composition is administered.

The term “prodrug” as used herein is intended to encompass a class of analogs of compounds of the present invention wherein a metabolically labile moiety is attached to said compound of the invention through an available NH, C(O)H, COOH, C(O)NH2, OH or SH functionality. The prodrug-forming moieties are removed by metabolic processes and release the active compounds having the free NH, C(O)H, COOH, C(O)NH2, OH, or SH group in vivo. Prodrugs are useful for adjusting such pharmacokinetic properties of the compounds as solubility and/or hydrophobicity, absorption in the gastrointestinal tract, bioavailability, tissue penetration, and rate of clearance. Design and preparation of such prodrugs are known to those skilled in the art, and are described in: Various forms of prodrugs are well known in the art and are described in:
a) The Practice of Medicinal Chemistry, Camille G. Wermuth et al., Ch. 31 (Academic Press, 1996).
b) Design of Prodrugs, edited by H. Bundgaard, (Elsevier, 1985); 33.
c) A Textbook of Drug Design and Development, P. Krogsgaard-Larson and H. Bundgaard, eds. Ch. 5, pp. 113-191 (Harwood Academic Publishers, 1991); and
d) Hydrolysis in Drug and Prodrug Metabolism, Bernard Testa and Joachim M. Mayer, (Wiley-VCH, 2003).
e) Prodrugs: challenges and rewards, Valentino J. Stella et al., Springer, 2007

Said references are incorporated herein by reference, particularly as to the description of prodrugs.

### General Methods for Preparation of Compounds

The present invention also provides a method for the synthesis of compounds of the present disclosure. The present invention further provides a method for the synthesis of compounds useful as intermediates in the preparation of compounds of the present disclosure. The compounds can be prepared according to the methods described below using readily available starting materials and reagents. In these reactions, variants may be employed, which are themselves known to those of ordinary skill in this art but are not described in detail here. Those skilled in the art of organic synthesis will appreciate that there exist multiple means of producing compounds of the present disclosure. Illustrative synthetic methods, including those directed to specific, selected compounds noted in Tables 1-14, are as set forth herein.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by one of ordinary skill in the art by routine optimization procedures.

One skilled in the art of organic synthesis understands that vulnerable moieties such as C(O)OH, C(O), and C(O)H, NH, C(O)NH2, OH, and SH moieties may be protected and deprotected, as necessary. Protecting groups for C(O)OH moieties include, but are not limited to, allyl, benzoylmethyl, benzyl, benzyloxymethyl, tert-butyl, ethyl, methyl, 2,2,2-trichloroethyl, and the like. Protecting groups for C(O) and C(O)H moieties include, but are not limited to, 1,3-dioxylketal, diethylketal, dimethylketal, 1,3-dithianylketal, O-methyloxime, O-phenyloxime, and the like. Protecting groups for NH moieties include, but are not limited to, acetyl, benzoyl, benzyl (phenylmethyl), benzylidene, benzyloxycarbonyl (Cbz), tert-butoxycarbonyl (Boc), 3,4-dimethoxybenzyloxycarbonyl, diphenylmethyl, diphenylphosphoryl, formyl, methanesulfonyl, para-methoxybenzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

A discussion of protecting groups is provided in T. H. Greene and P. G. M. Wuts, Protective Groups in Organic Synthesis, 3rd Ed., John Wiley & Sons, New York (1999).

The invention will now be further described with reference to the following illustrative examples in which, unless stated otherwise: (i) temperatures are given in degrees Celsius (° C.); operations are carried out at room temperature (RT) or ambient temperature, that is, in a range of 18-25° C.; (ii) organic solutions were dried over anhydrous sodium or magnesium sulfate unless otherwise stated; evaporation of organic solvent was carried out using a rotary evaporator under reduced pressure; (iii) column chromatography means flash chromatography on silica gel; thin layer chromatography (TLC) was carried out on silica gel plates; (iv) in general, the course of reactions was followed by TLC or liquid chromatography/mass spectroscopy (LC/MS) and reaction times are given for illustration only; (v) final products have satisfactory proton nuclear magnetic resonance (NMR) spectra and/or mass spectra data; (vi) yields are given for illustration only and are not necessarily those which can be obtained by diligent process development; preparations were repeated if more material was required; (vii) when given, NMR data is in the form of delta values for major diagnostic protons, given in part per million (ppm) relative to tetramethylsilane (TMS) as an internal standard, and were obtained in the solvent indicated; (viii) chemical symbols have their usual meanings; (ix) in the event that the nomenclature assigned to a given compound does not correspond to the compound structure depicted herein, the structure will control; (x) solvent ratio is given in volume:volume (v/v) terms. The chemical names of the compounds and drawing of the chemical formulas are produced using ChemOffice professional. The abbreviation for chemicals, solvents, reagents, and protecting groups are used as a common practice in the chemistry field.

Compounds of claim 1 of the current invention can be prepared according to Schemes A-D, as set forth below.

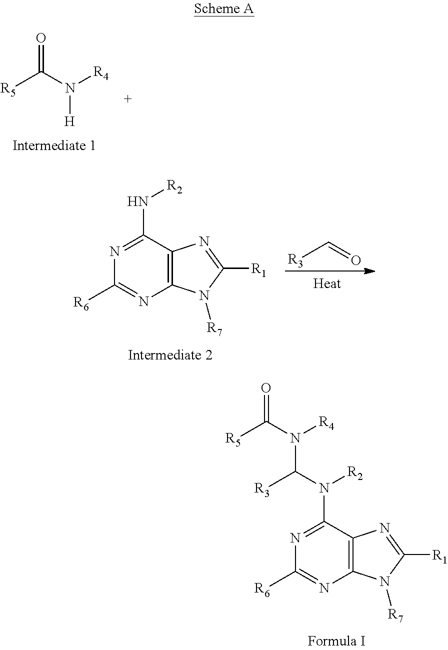

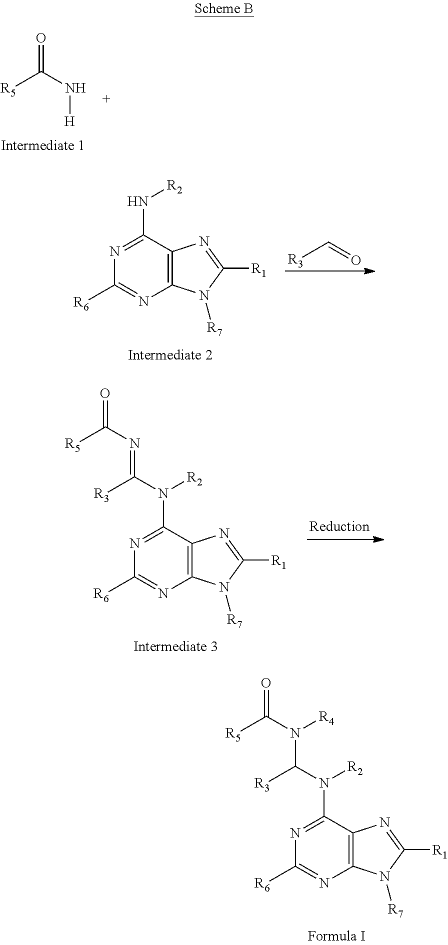

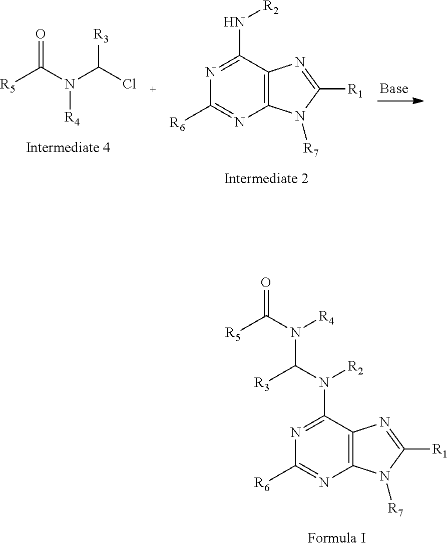

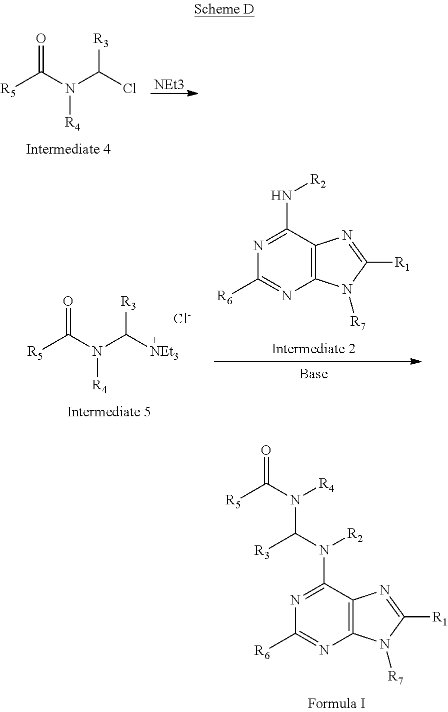

R₁, R₂, R₃, R₄, R₅, R₆, and R₇ in the above schemes are as defined above.

Intermediates 1, 2, and 4 can be commercially available or can be prepared by using synthetic methods known in the literature.

It is appreciated and understand that functionality on any of the R₁, R₂, R₃, R₄, R₅, R₆, and R₇ may be required to be protected to carry out reaction(s) and then finally deprotected to obtain the final product. The final compounds may be obtained in the racemic mixture, and then each constituent isomers can be purified by either crystallization or chiral chromatography.

### EXAMPLES

### Example 1

### Synthesis of N-(((2-chloro-9-((1S,2R,3S,4R,5R)-3,4-dihydroxybicyclo [3.1.0] hexan-2-yl)-9H-purin-6-yl) (dicyclopropylmethyl)amino) methyl) benzamide (T1-1)

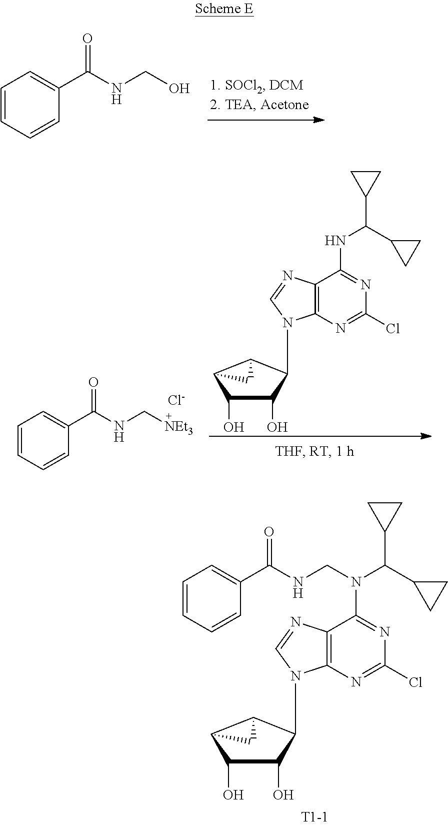

### Step 1: Synthesis of Benzamidomethyl Triethyl Ammonium Chloride

To a stirred solution of N-(hydroxymethyl) benzamide (6 g, 39.7 mmol) in DCM, thionyl chloride (8.7 mL, 119.2. mmol) was added dropwise at 10° C., the resulting reaction mixture stirred at RT for 2 h. The resulting clear solution was poured into heptanes, during this white precipitate (N-(chloromethyl) benzamide) was formed, filtered and washed with heptanes (50 mL) and dried under nitrogen. N-(chloromethyl) benzamide (7.2 g, 42.6 mmol) was immediately dissolved in dry acetone, and Et₃N (6 mL, 42.6 mmol) was added dropwise at RT. An additional 100 mL of dry acetone was added to the resulting white thick suspension, stirred for 30 min, the reaction mixture was filtered under a high vacuum and washed with acetone, dried to give a colorless powder. Which was further recrystallized from chloroform to give benzamidomethyl triethyl ammonium chloride as a white crystal.

% Yield: 3.6 g (32%)

¹H NMR (400 MHz, CDCl3): δ 10.81-10.78 (m, 1H), 8.36-8.33 (m, 2H), 7.57-7.47 (m, 3H), 5.01-4.9 (m, 2H), 3.4-3.34 (m, 6H), 1.52-139 (m, 6H).

### Step 2: Synthesis of N-(((2-chloro-9-((1S,2R,3S,4R,5R)-3,4-dihydroxybicyclo [3.1.0] hexan-2-yl)-9H-purin-6-yl) (dicyclopropylmethyl)amino) methyl) benzamide (T1-1)

To a stirred solution of (1R,2R,3S,4R,5S)-4-(2-chloro-6-((dicyclopropylmethyl)amino)-9H-purin-9-yl) bicyclo [3.1.0] hexane-2,3-diol (300 mg, 0.8 mmol) in dry THF (15 mL). Benzamidomethyl triethyl ammonium chloride (2.184 g, 8 mmol) was added at RT, the resulting reaction mixture was stirred for 24 h. After completion of the reaction by TLC, reaction mixture was concentrated under reduced pressure to dryness. The crude residue was purified from preparative HPLC to give the desired compound (T1-1) as an off-white Solid.

1H NMR (400 MHz, DMSO-d6): VT δ 8.99 (m, 1H), 8.07-8.05 (m, 1H), 7.87-7.84 (m, 2H) 7.79-7.75 (m, 2H), 7.54-7.41 (m, 5H), 4.89-4.80 (m, 3H), 4.6-4.57 (m, 3H), 4.13 (m, 1H), 1.92-1.88 (m, 1H), 1.57-1.54 (m, 1H), 1.19-1.15 (m, 3H), 0.72-0.69 (m, 1H), 0.51-0.47 (m, 2H), 0.32-0.37 (m, 6H).

Following compounds in Tables 1-4 can be made using a procedure similar to those outlined the schemes A-D.

TABLE 1

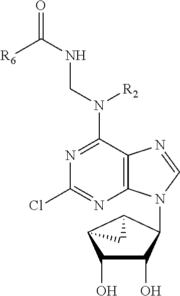

Formula T1

| Comp. No. | R₂ | R₆ |
|---|---|---|
| T1-1 | 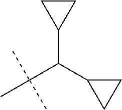 | 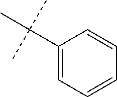 |
| T1-2 | 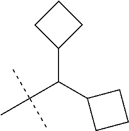 | 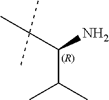 |
| T1-3 | 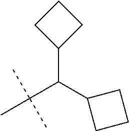 | —CH₃ |
| T1-4 | 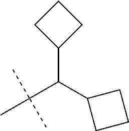 | —CH₂CH₃ |
| T1-5 | 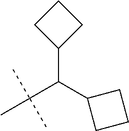 | 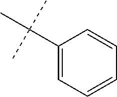 |
| T1-6 | 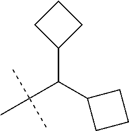 | 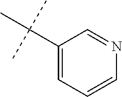 |
| T1-7 | 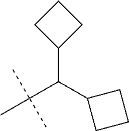 | 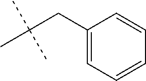 |
| T1-8 | 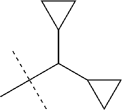 | 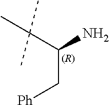 |
| T1-9 | 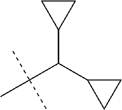 | 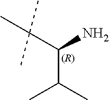 |
| T1-10 | 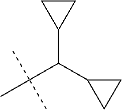 | —CH₃ |
| T1-11 | 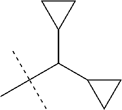 | —CH₂CH₃ |
| T1-12 | 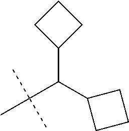 | 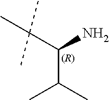 |
| T1-13 | 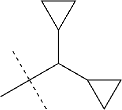 | 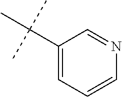 |
| T1-14 | 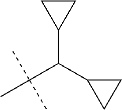 | 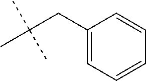 |
| T1-15 | 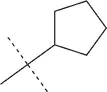 | 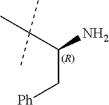 |
| T1-16 | 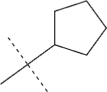 | 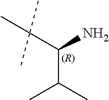 |
| T1-17 | 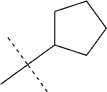 | —CH₃ |
| T1-18 | 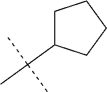 | —CH₂CH₃ |
| T1-19 | 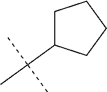 | 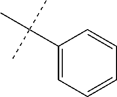 |
| T1-20 | 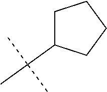 | 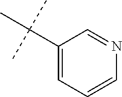 |
| T1-21 | 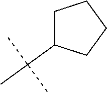 | 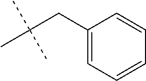 |
| T1-22 | H | 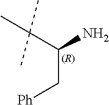 |
| T1-23 | H | 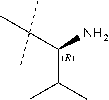 |
| T1-24 | H | —CH₃ |
| T1-25 | H | —CH₂CH₃ |
| T1-26 | H | 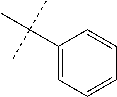 |
| T1-27 | H | 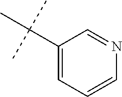 |
| T1-28 | H | 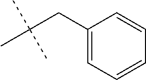 |

TABLE 2

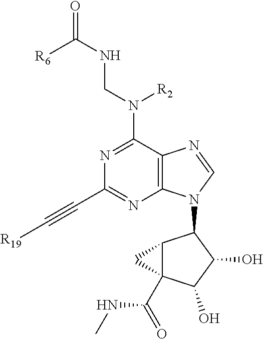

Formula T2

| Comp. No. | R₂ | R₆ | R₁₉ |
|---|---|---|---|
| T2-1 | 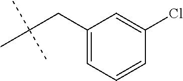 | 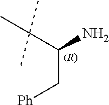 | 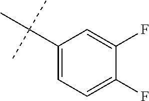 |
| T2-2 | 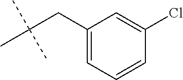 | 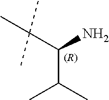 | 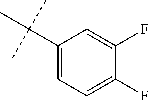 |
| T2-3 | 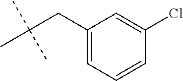 | —CH₃ | 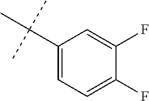 |
| T2-4 | 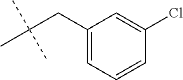 | —CH₂CH₃ | 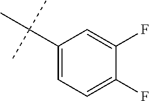 |
| T2-5 | 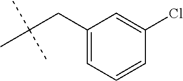 | 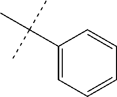 | 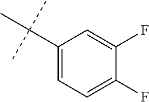 |
| T2-6 | 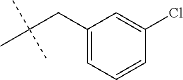 | 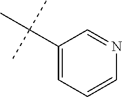 | 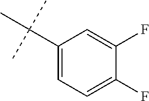 |
| T2-7 | 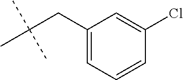 | 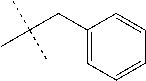 | 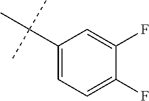 |
| T2-8 | 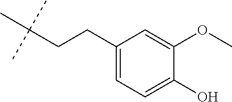 | 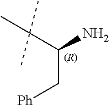 | 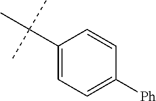 |
| T2-9 | 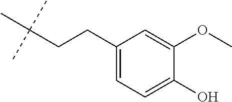 | 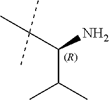 | 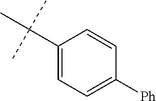 |
| T2-10 | 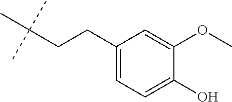 | —CH₃ | 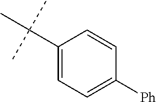 |
| T2-11 | 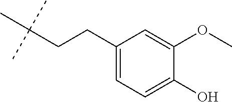 | —CH₂CH₃ | 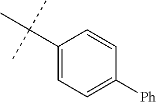 |
| T2-12 | 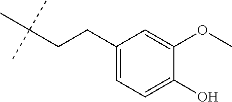 | 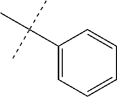 | 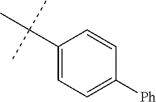 |
| T2-13 | 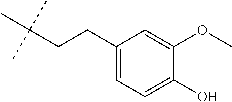 | 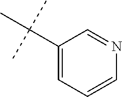 | 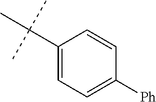 |
| T2-14 | 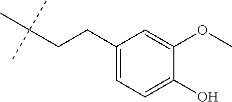 | 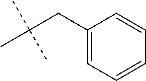 | 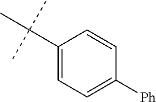 |

TABLE 3

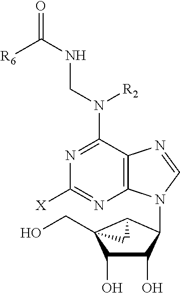

Formula T3

| Comp. No. | R₂ | R₆ | X |
|---|---|---|---|
| T3-1 | 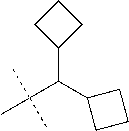 | 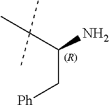 | Cl |
| T3-2 | 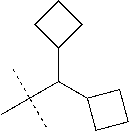 | 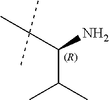 | Cl |
| T3-3 | 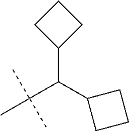 | —CH₃ | Cl |
| T3-4 | 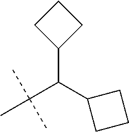 | —CH₂CH₃ | Cl |
| T3-5 | 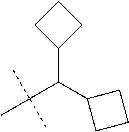 | 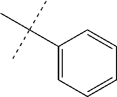 | Cl |
| T3-6 | 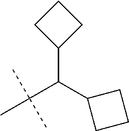 | 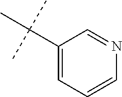 | Cl |
| T3-7 | 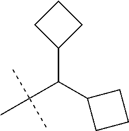 | 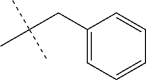 | Cl |
| T3-1 | 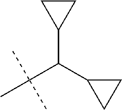 | 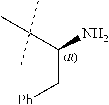 | Cl |
| T3-8 | 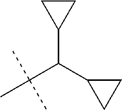 | 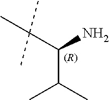 | Cl |
| T3-9 | 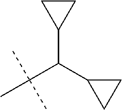 | —CH₃ | Cl |
| T3-10 | 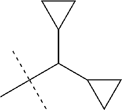 | —CH₂CH₃ | Cl |
| T3-11 | 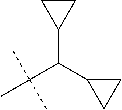 | 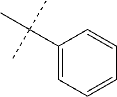 | Cl |
| T3-12 | 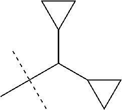 | 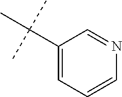 | Cl |
| T3-13 | 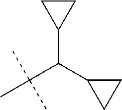 | 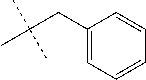 | Cl |
| T3-14 | 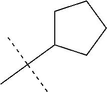 | 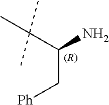 | H |
| T3-15 | 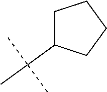 | 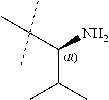 | H |
| T3-16 | 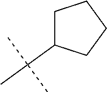 | —CH₃ | H |
| T3-17 | 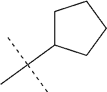 | —CH₂CH₃ | H |
| T3-18 | 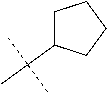 | 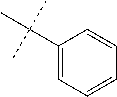 | H |
| T3-19 | 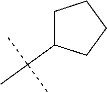 | 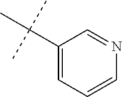 | H |
| T3-20 | 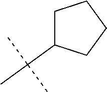 | 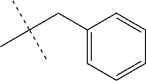 | H |

### Example 2

Pharmacokinetic Analysis of Compound T1-1 for Determination Utility as a Prodrug of MRS5474:

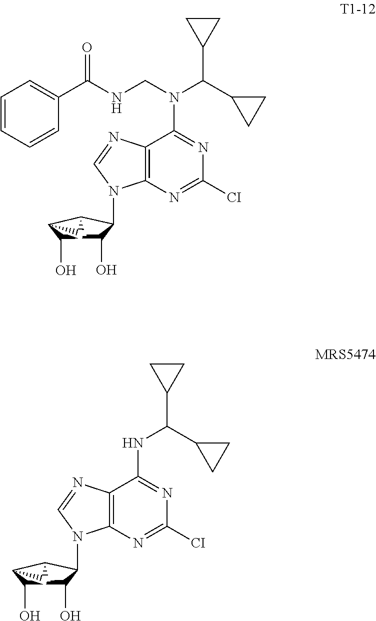

Preparation of Dose Solutions:

The dosing formulation (for i.v.) of T1-1 for mouse PK studies were prepared as 0.27 mg/mL solution of 10% DMSO+50% PEG400+40% WATER (Dissolved 1.19 mg of T1-1 in 0.441 mL of DMSO with vortexing and sonication, then added 2.204 mL of PEG400 and 1.763 mL of H2O with vortexing to obtain a solution). The dosing formulation (for p.o.) of T1-1 for mouse PK studies were prepared 2.71 mg/mL solution of 10% DMSO+50% PEG400+40% WATER (Dissolved 10.17 mg of in 0.375 mL of DMSO with vortexing and sonication, then added 1.876 mL O21+I21 of PEG400 and 1.501 mL of H2O with vortexing to obtain a solution.)

Animals: Male CD-1 mice (6-8 weeks of age), weighing 20-22 g; all animals were allowed to acclimate to this environment for one week prior to experimental manipulations.

Study Design: Study design The CD1 mice were given a single dose of T1-1 by bodyweight via intravenous (i.v.), or via oral gavage (p.o) administration. The blood and brain tissue samples were collected at intervals (h) of 0, 0.5, 1, 2, 4, 8, and 24. (n=3, each time point).

Plasma Sample Preparation: Approximately 0.3 mL blood will be collected at each time point. After collecting the blood at each time point, add 250 to 300 μL of the blood sample to Eppendorf tube containing EDTA solution+10 μL of inhibitor stock and vortexed for 15 minutes and then centrifugation at 4000 g for 5 minutes in a 4° C. centrifuge to obtain plasma.

The desired serial concentrations of working solutions were achieved by diluting a stock solution of the analyte with 50% acetonitiile in water solution. 5 μL of working solutions (5, 10, 20, 50, 100, 500, 1000, 5000, 10000 ng/mL) were added to 50 μL of the blank CD1 Mouse plasma to achieve calibration standards of 0.5-1000 ng/mL (0.5, 1, 2, 5, 10, 50, 100, 500, 1000 ng/mL) in a total volume of 55 μL. Five quality control samples at 1 ng/mL, 2 ng/mL, 5 ng/mL, 50 ng/mL, and 800 ng/mL for plasma were prepared independently of those used for the calibration curves. These QC samples were prepared on the day of analysis in the same way as calibration standards. 55 μL standards, 55 μL QC samples and 55 unknown samples (50 μL plasma with 5 μL blank solution) were added to 200 μL of acetonitrile containing IS mixture for precipitating protein respectively. Then the samples were vortexed for 30 s. After centrifugation at 4 degree Celsius, 3900 rpm for 15 min, the supernatant was diluted 3 times with water. 1 μL of diluted supernatant was injected into the LC/MS/MS system for quantitative analysis using (Shimadzu-DGU-20A5 integrated with AB AP15500 LCMS/MS instrument) with to determine plasma concentrations of T1-1 and the metabolite (parent compound) MRA5474. The data were analyzed for test article T1-1 and the metabolite (parent compound) MRA5474 for PK profile.

The mobile phase consisted of acetonitrile and water mixtures, solution A: 5% acetonitrile (0.1% formic acid) and solution B: 95% acetonitrile (0.1% formic acid) with gradient elution from 90 to 10% solution A over 1.71 min. The flow rate was 0.7 mL/min. YMC Triart C18 5 μm (50×2.1 mm) column was used for the separation of the sample components.

Results: Summary of IV and PO of Plasma Pharmacokinetic Parameters

| | | T1-1 | T1-1 | MRS5474 | MRS5474 |
|---|---|---|---|---|---|
| Dose(mg/kg): | | 1.35 | 13.55 | 1.35 | 13.55 |
| PK parameters | Unit | IV(plasma) | PO(plasma) | IV(plasma) | PO(plasma) |
| Cl_obs | mL/min/kg | 56.98 | NA | | |
| T1/2 | h | 0.29 | NA | 1.55 | 2.32 |
| C0 | ng/mL | 613.37 | NA | | |
| Cmax | ng/mL | NA | 132.00 | 127.00 | 522.00 |
| Tmax | h | NA | 0.50 | 1.00 | 1.00 |
| AUClast | h*ng/mL | 391.19 | 467.10 | 313.07 | 6986.22 |
| AUCInf | h*ng/mL | 394.88 | NA | 321.48 | 6991.53 |
| AUC_% Extrap_obs | % | 0.93 | NA | 2.62 | 0.08 |
| MRTInf_obs | h | 0.46 | NA | 2.32 | 6.36 |
| AUClast/D | h*mg/mL | 289.77 | 34.47 | | |
| Vss_obs | L/kg | 1.57 | NA | | |
| F | % | NA | 11.90 | | |

Quantitative Real-Time PCR Analysis

Male CD1 mice were administered vehicle and test compound (T1-1) by oral gavage. After 2 hours, the were decapitated, and their brain samples were collected. Total RNA from the brain tissue were isolated using denaturing agarose gel electrophoresis, RNA integrity was confirmed, and the concentration was quantified by measuring the optical density. Reverse transcription was performed (Invitrogen) for cDNA synthesis according to the manufacturer's instructions. Real-time qPCR was performed on Real-Time PCR System using SYBR Green. The amplification reactions were performed as per the supplier's protocol.

qPCR Results (Actin):

| | | | | | | | | | Relative mRNA | |
|---|---|---|---|---|---|---|---|---|---|---|
| | | | Target | Actin | Target | Actin | ΔCt(Target-Actin) | | expression(2^{(-ΔCt)}) | |
| Gene | Treatment | ID | CT(n = 1) | | CT(n = 2) | | n = 1 | n = 2 | n = l | n = 2 |
| Homer 1a | Vehicle | M1 | 17.57 | 15.92 | 17.67 | 16.02 | 1.65 | 1.66 | 0.32 | 0.32 |
| | | M2 | 17.18 | 16.17 | 17.19 | 16.24 | 1.01 | 0.95 | 0.50 | 0.52 |
| | | M3 | 17.18 | 15.98 | 17.15 | 15.89 | 1.20 | 1.26 | 0.43 | 0.42 |
| | T1-1:PO(2 h) | M31 | 17.65 | 16.64 | 17.69 | 16.82 | 1.05 | 0.87 | 0.48 | 0.55 |
| | | M32 | 17.97 | 16.56 | 17.79 | 16.61 | 1.41 | 1.18 | 0.38 | 0.44 |
| | | M33 | 17.16 | 15.81 | 17.18 | 15.36 | 1.35 | 1.33 | 0.39 | 0.40 |
| | | | | Relative | | | | | | |
| | | | | mRNA | | | | | | |
| | | | | expression | | | | | | |
| | | | | (2^{(-ΔCt)}) | % Vehicle | | | | | |
| | Gene | Treatment | ID | Average | n = l | n = 2 | Average | Mean | SEM | TTEST |
| | Homer 1a | Vehicle | M1 | 0.32 | 81.67 | 81.37 | 31.52 | 100.00 | 8.17 | 1.00 |
| | | | M2 | 0.51 | 127.40 | 133.06 | 130.23 | | | |
| | | | M3 | 0.43 | 111.41 | 107.31 | 109.36 | | | |
| | | T1-1:PO(2 h) | M31 | 0.51 | 123.98 | 139.98 | 131.98 | 112.74 | 9.67 | 0.36 |
| | | | M32 | 0.41 | 96.37 | 113.39 | 104.88 | | | |
| | | | M33 | 0.40 | 100.64 | 102.10 | 101.37 | | | |

qPCR Results (GAPDH):

| | | | | | | | | | Relative mRNA | |
|---|---|---|---|---|---|---|---|---|---|---|
| | | | Target | GAPDH | Target | GAFDH | ACtTarget-Actin) | | expression(2^{(-ΔCt)}) | |
| Gene | Treatment | ID | CT(n = 1) | | CT(n = 2) | | n = 1 | n = 2 | n = 1 | n = 2 |
| Homer 1a | Vehicle | M1 | 17.57 | 14.65 | 17.67 | 14.47 | 2.92 | 3.21 | 0.1322 | 0.1081 |
| | | M2 | 17.18 | 14.42 | 17.19 | 14.46 | 2.76 | 2.73 | 0.1475 | 0.1503 |
| | | M3 | 17.18 | 14.32 | 17.15 | 14.36 | 2.86 | 2.79 | 0.1377 | 0.1442 |
| | T1-1:PO(2 h) | M31 | 17.69 | 14.56 | 17.69 | 14.67 | 3.13 | 3.02 | 0.1140 | 0.1234 |
| | | M32 | 17.97 | 14.28 | 17.79 | 14.21 | 3.69 | 3.57 | 0.0774 | 0.0840 |
| | | M33 | 17.16 | 14.02 | 17.18 | 14.09 | 3.14 | 3.09 | 0.1137 | 0.1171 |
| | | | | Relative | | | | | | |
| | | | | mRNA | | | | | | |
| | | | | expression | | | | | | |
| | | | | (2^{(-ΔCt)}) | % Vehicle | | | | | |
| | Gene | Treatment | ID | Average | n = l | n = 2 | Average | Mean | SEM | TTEST |
| | Homer 1a | Vehicle | M1 | 0.1201 | 83.80 | 68.54 | 76.17 | 100.00 | 7.08 | 1.00 |
| | | | M2 | 0.1489 | 93.59 | 95.29 | 94.44 | | | |
| | | | M3 | 0.1409 | 87.28 | 91.42 | 89.35 | | | |
| | | T1-1:PO(2 h) | M31 | 0.1187 | 72.27 | 78.27 | 75.27 | 66.54 | 7.71 | 0.02 |
| | | | M32 | 0.0807 | 49.10 | 53.24 | 51.17 | | | |
| | | | M33 | 0.1154 | 72.10 | 74.26 | 73.18 | | | |

1. A compound of Formula I or its pharmaceutically acceptable salt thereof or a prodrug thereof or metabolite thereof, or composition thereof 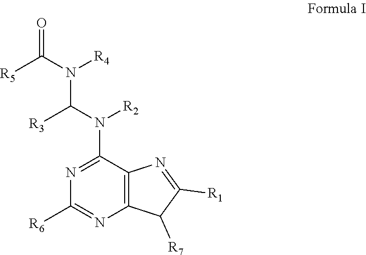
wherein
R₁ is selected from hydrogen, alkyl, alkenyl, alkynyl, aryl, heteroaryl wherein said alkyl, alkenyl, alkynyl, aryl, heteroaryl each are optionally substituted with one or more R₈;
R₂, R₅, and R₇ is selected from hydrogen, amino acid residue, alkyl, alkenyl, alkynyl, heteroalkyl, aryl, arylalkyl, heteroaryl, heteroarylalkyl, cycloalkyl, bicycloalkyl, and heterocycloalkyl wherein said amino acid, alkyl, alkenyl, alkynyl, heteroalkyl, aryl, arylalkyl, heteroaryl, heteroarylalkyl, cycloalkyl, bicycloalkyl, and heterocycloalkyl each is optionally substituted with one or more R₈;
R₃, and R₄ are independently from hydrogen and alkyl, wherein said alkyl is optionally substituted with one or more R₈;
R₆ is selected from hydrogen, halogen, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each are optionally substituted with one or more R₈;
R₈ is independently selected for each occurrence from the group consisting of hydrogen, amino acid, lower alkyl, lower alkenyl, lower alkynyl, cycloalkyl, heterocycloalkyl aryl, heteroaryl, alicyclic, arylalkyl, heteroarylalkyl, lower alkoxy, aryloxy, amino, alkylamino, dialkylamino, diarylalkylamino, alkylthio, arylthio, heteroarylthio, oxo, oxa, —(CH₂)ₙOR₉, —C(O)—R₉, —C(O)OR₉, —C(O)NR₉R₁₀, —C(S)NR₉R₁₀, CO₂H, acyloxy, halo, —CN, —NO₂, —N₃, —SH, —OH, —CH(CF₃)NR₉R₁₀, —C(═N)NR₉, perhaloalkyl, perhaloalkoxy, perhaloacyl, guanidinyl, pyridinyl, thiophene, furanyl, indolyl, indazolyl, phosphonate, phosphonic acid, mono, or poly-phosphate derivative, phosphoramide, sulfonate, sulfone, sulfate, sulphonamide, carbamate, urea, thiourea, thioamide, and thioalkyl; wherein R₈, R₉ and R₁₀ are independently selected from hydrogen, methyl, ethyl, and benzyl.

2. The compound according to claim 1 wherein
R₁ is hydrogen,
R₂ is selected from hydrogen, alkyl, heteroalkyl, arylalkyl, heteroaryl, heteroarylalkyl, cycloalkyl, bicycloalkyl, and heterocycloalkyl wherein said alkyl, heteroalkyl, arylalkyl, heteroaryl, heteroarylalkyl, cycloalkyl, bicycloalkyl, and heterocycloalkyl each are optionally substituted with one or more R₈,
R₃ is independently selected from hydrogen, methyl, ethyl, isopropyl and phenyl.
R₄ is selected from hydrogen and alkyl wherein said alkyl is optionally substituted with R₈.
R₅ is selected amino acid residue, alkyl, heteroalkyl, aryl, arylalkyl, heteroaryl, heteroarylalkyl, wherein said amino acid residue, alkyl, heteroalkyl, aryl, arylalkyl, heteroaryl, heteroarylalkyl, each are optionally substituted with one or more R₈ and
R₆ is selected from hydrogen, halogen, alkyl, aryl, heteroaryl, and alkynyl-aryl wherein said alkyl, aryl, heteroaryl, alkynyl-aryl each are optionally substituted with one or more R₈.

3. The compound according to claim 1 wherein R₆ is selected from Formula IIA and Formula IIB 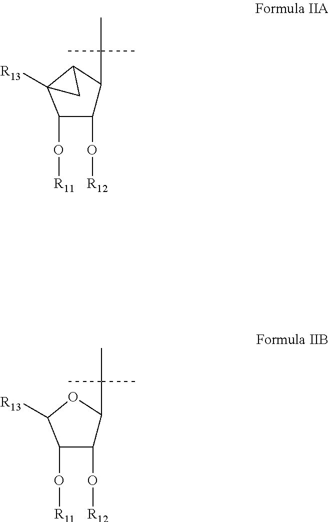
wherein
R₁₁ and R₁₂ are independently selected from hydrogen, alkyl, amino acid residue, —C(O)R₁₄, —C(O)NR₁₄R₁₅ wherein R₁₄ and R₁₅ are independently selected from hydrogen, alkyl wherein said alkyl and aryl each are optionally substituted with one or more R₈; and R₁₃ is selected from hydrogen, —CH₂—OH, —CH₂—Cl, —COOR₁₆, CONR₁₆R₁₇ wherein R₁₆ and R₁₇ are independently selected from hydrogen, alkyl wherein said alkyl and aryl each are optionally substituted with one or more R₈.

4. The compound according to claim 1 wherein R₆ is selected from Formula IIIA and Formula IIIB. 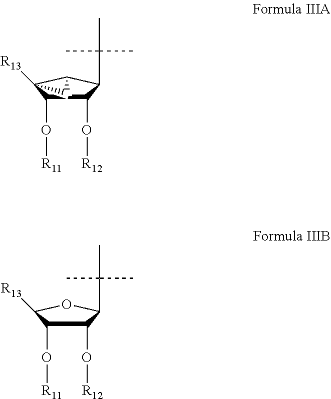
Wherein R₁₁ and R₁₂ are as defined in claim 4

5. The compound according claim 1 wherein R₆ is Formula IIIA.

6. The compound according claim 1 wherein R₆ is Formula IIIB

7. The compound according to claim 1, having the structure of Formula IV 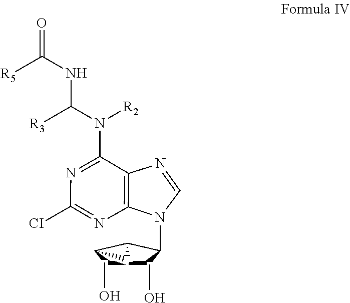
wherein R₂, R₃, and R₅ are as defined in claim 1.

8. The compound according to claim 1, having the structure of Formula V 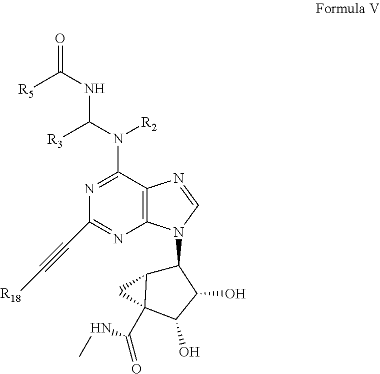
wherein R₁₈ is selected from aryl or heteroaryl which is optionally substituted with one or more R₈; and R₂, R₃, and R₅ are as defined in claim 1.

9. The compound according to claim 1, having the structure of Formula VIA and Formula VIB 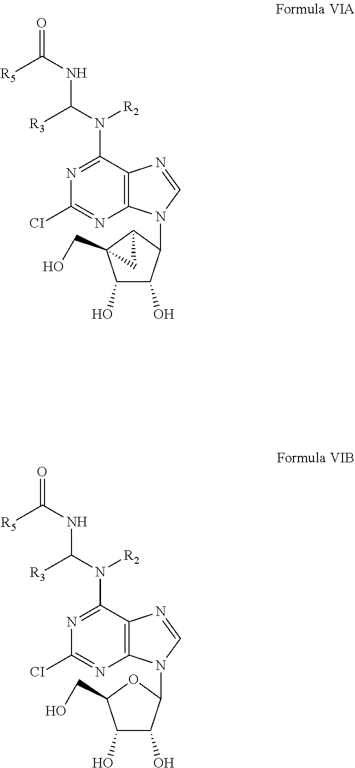
wherein R₂, R₃, and R₅ are as defined in claim 1.

10. The compound according to claim 1, having the structure of Formula VII. 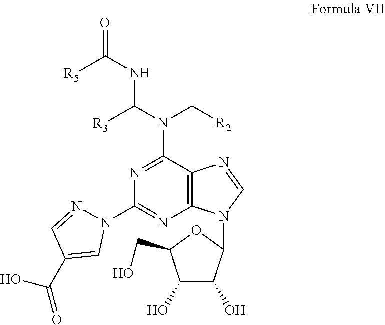
wherein R₂, R₃, and R₅ are as defined in claim 1.

11. The compound according to claim 1 and having any of the structure of Tables 1-4 or its pharmaceutically acceptable salt thereof or a prodrug thereof or metabolite thereof, or composition thereof.

12. The compound according to claim 1 and having any of the structure Table 1 or its pharmaceutically acceptable salt thereof or a prodrug thereof or metabolite thereof, or composition thereof.

13. The compound according to claim 1 and having any of the structure Table 2 or its pharmaceutically acceptable salt thereof or a prodrug thereof or metabolite thereof, or composition thereof.

14. The compound according to claim 1 and having any of the structure Table 3 or its pharmaceutically acceptable salt thereof or a prodrug thereof or metabolite thereof, or composition thereof.

15. The compound according to claim 1 and having any of the structure of Table 4 or its pharmaceutically acceptable salt or a prodrug or metabolite thereof.

16. A pharmaceutical composition comprising a compound of Formula I as defined in any of the claim 1 or a pharmaceutically acceptable salt, ester or prodrug or metabolite thereof in association with a pharmaceutically acceptable diluent or carrier to form a formulation system for delivering the compound.

17. The pharmaceutical composition of claim 16 that is a modified-release formulation comprising a compound of Formula I a single active pharmaceutical ingredient, (a) at least one release controlling polymer selected from the group consisting of pH-dependent polymers and non-pH-dependent polymers, and (b) at least one stabilizer selected from the group consisting of acidifying agents and hydrophobizing agents, wherein the total amount of water content in the formulation is not more than 5% by weight of the formulation.

18. A method of use of a compound of Formula I as defined in any of the claim 1 or a pharmaceutically acceptable salt, ester or prodrug or metabolite thereof as a prodrug of the corresponding parent active compound for therapeutic use.

19. The method of use according to claim 18 using a compound of Formula I as defined in claim 1 or a pharmaceutically acceptable salt, ester or prodrug or metabolite thereof in the preparation of a medicament for the treatment of cardiovascular disease, nervous system disorders, Parkinson's disease, pain, neurodegenerative disorders, ischemia and neuroprotection, sleep, renal system disorders, pulmonary disorders, inflammatory disorders, endocrine disorders, cancer, and visual disorders.

20. A process of producing a compound of Formula I as defined in any of the claim 1 or its pharmaceutically acceptable salt or prodrug or metabolite.
